(19) United States
(12) Patent Application Publication
(10) Pub. No.: US 2012/0203295 A1
(43) Pub. Date: Aug. 9, 2012

(54) PACING SITE OPTIMIZATION USING PACED INTERVENTRICULAR DELAYS

(76) Inventors: Barun Maskara, Blaine, MN (US); Keith L. Herrmann, Minneapolis, MN (US)

(21) Appl. No.: 13/368,008

(22) Filed: Feb. 7, 2012

Related U.S. Application Data
(60) Provisional application No. 61/440,550, filed on Feb. 8, 2011.

(51) Int. Cl. A61N 1/365

(52) U.S. Cl. 607/17

(57) ABSTRACT

An apparatus comprises a cardiac signal sensing circuit, a stimulus circuit, and a control circuit. The control circuit includes a pacing site locating circuit that initiates a first electrical stimulus from a first electrode positioned in or near a first ventricle of a heart, determines a first time interval between delivery of the first electrical stimulus and a subsequent cardiac event sensed at a selectable electrode location in or near a second ventricle of the heart, initiates a second electrical stimulus at the selectable electrode location in or near the second ventricle, determines a second time interval between delivery of the second electrical stimulus and a subsequent cardiac event sensed at the first ventricle, calculates a difference between the first and second time intervals, and generates an indication of a preferred pacing site in the second ventricle according to the calculated differences between the first and second time intervals.

### CROSS-REFERENCE TO RELATED APPLICATION

This application claims the benefit of U.S. Provisional Application No. 61/440,550, filed on Feb. 8, 2011, under 35 U.S.C. §119(e), which is hereby incorporated by reference in its entirety.

### BACKGROUND

Medical devices include devices designed to be implanted into a patient. Some examples of these implantable medical devices (IMDs) include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients or subjects using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of IMDs include implantable diagnostic devices, implantable drug delivery systems, or implantable devices with neural stimulation capability.

Some IMDs detect events by monitoring electrical heart activity' signals. In CFM devices, these events can include heart chamber expansions or contractions. By monitoring cardiac signals indicative of expansions or contractions, IMDs can detect abnormally slow heart rate, or bradycardia. In response to an abnormally slow heart rate some CFM devices deliver electrical pacing stimulation energy to induce cardiac depolarization and contraction. The pacing stimulation energy is delivered to provide a depolarization rate that improves hemodynamic function of the patient.

Delivery of pacing therapy should be optimized to ensure therapy delivery and yet avoid unnecessary stress on the heart and unnecessary reduction of battery life. Optimal selection of the site for delivery of the pacing therapy can be part of pacing therapy optimization. Optimal site selection can lead to optimized use of pacing energy and to improved hemodynamic function of the patient or subject.

An example of optimizing pacing therapy using evoked response and propagation delay can be found in Min et al., Optimization of Cardiac Pacing Based on Propagation Delay,” U.S. Patent Application Publication No. US 2010/0121401, filed Nov. 7, 2008. An example of a method to improve patient response to cardiac resynchronization therapy (CRT) can be found in Park et al., “System and Method for Improving CRT Response and Identifying Potential Non-Responders to CRT Therapy,” U.S. Patent Application Publication No. US 2008/0306567, filed Jun. 7, 2007.

### Overview

This document relates generally to systems, devices, and methods that provide electrical pacing therapy to the heart of a patient or subject. In particular it relates to, systems, devices, and methods that determine a preferred site of the heart to provide pacing therapy.

An apparatus example includes a cardiac signal sensing circuit, a stimulus circuit, and a control circuit. The control circuit includes a pacing site locating circuit that initiates a first electrical stimulus from a first electrode positioned in or near a first ventricle of a heart, determines a first time interval between delivery of the first electrical stimulus and a subsequent cardiac event sensed at a selectable electrode location in or near a second ventricle of the heart, initiates a second electrical stimulus at the selectable electrode location in or near the second ventricle, determines a second time interval between delivery of the second electrical stimulus and a subsequent cardiac event sensed at the first ventricle, calculates a difference between the first and second time intervals, iteratively select one or more additional electrodes located in or near the second ventricle, and generates an indication of a preferred pacing site in the second ventricle according to the calculated differences between the first and second time intervals.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 is an illustration of an example of portions of a system that includes an IMD.

FIG. 2 is an illustration of portions of another system that uses an IMD.

FIG. 3 is a flow diagram of an example of a method of operating a medical device.

FIG. 4 illustrates an example of determining time intervals between cardiac events.

FIG. 5 is a block diagram of portions of an example of a medical device that can determine a preferred site to deliver pacing therapy.

### DETAILED DESCRIPTION

An implantable medical device (IMD) may include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable or partially implantable device need not include all of the features described herein, but may be implemented to include selected features that provide for unique structures or functionality. Such a device may be implemented to provide a variety of therapeutic or diagnostic functions.

As explained previously, pacing therapy should be optimized for a patient. This can include optimizing one or both of the pacing site and the pacing energy used to deliver the therapy. The pacing site selected to deliver the pacing stimulus can have a significant impact on the therapy. An improper pacing site (e.g., a pacing site too close to scar tissue) can lead to slow activation of myocardial tissue. Slow activation can lead to a caregiver programming an IMD to deliver a higher level of energy in the pacing stimulation than is necessary or can lead to a sub-optimal hemodynamic response to the pacing therapy. Thus, finding an optimal pacing site can be part of optimizing pacing therapy.

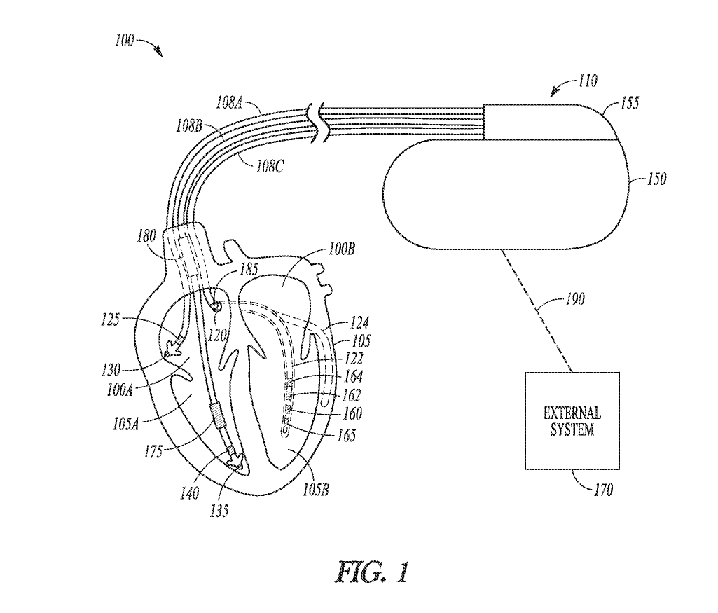

FIG. 1 is an illustration of portions of a system that uses an IMD 110. Examples of IMD 110 include, without limitation, a pacemaker, a defibrillator, a cardiac resynchronization therapy (CRT) device, or a combination of such devices. The system 100 also typically includes an IMD programmer or other external device 170 that communicates wireless signals 190 with the IMD 110, such as by using radio frequency (RF) or other telemetry signals.

The IMD 110 can be coupled by one or more leads 108A-C to heart 105. Cardiac leads 108A-C include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals.

Sensed electrical cardiac signals can be sampled to create an electrogram. An electrogram can be analyzed by the IMD and/or can be stored in the IMD and later communicated to an external device where the sampled signals can be displayed for analysis.

Heart 105 includes a right atrium 100A, a left atrium 100B, a right ventricle 105A, a left ventricle 105B, and a coronary sinus 120 extending from right atrium 100A. Right atrial (RA) lead 108A includes electrodes (electrical contacts, such as ring electrode 125 and tip electrode 130) disposed in an atrium 100A of heart 105 for sensing signals, or delivering pacing therapy, or both, to the atrium 100A.

Right ventricular (RV) lead 108B includes one or more electrodes, such as tip electrode 135 and ring electrode 140, for sensing signals, delivering pacing therapy, or both sensing signals and delivering pacing therapy. Lead 108B optionally also includes additional electrodes, such as for delivering atrial cardioversion, atrial defibrillation, ventricular cardioversion, ventricular defibrillation, or combinations thereof to heart 105. Such electrodes typically have larger surface areas than pacing electrodes in order to handle the larger energies involved in defibrillation. Lead 108B optionally provides resynchronization therapy to the heart 105. Resynchronization therapy is typically delivered to the ventricles in order to better synchronize the timing of depolarizations between ventricles.

The IMD 110 can include a third cardiac lead 108C attached to the IMD 110 through the header 155. The third cardiac lead 108C includes electrodes 160, 162, 164, and 165 placed in a coronary vein lying epicardially on the left ventricle (LV) 105B via the coronary vein. The third cardiac lead 108C may include anywhere from two to eight electrodes, and may include a ring electrode 185 positioned near the coronary sinus (CS) 120.

Lead 108B can include a first defibrillation coil electrode 175 located proximal to tip and ring electrodes 135, 140 for placement in a right ventricle, and a second defibrillation coil electrode 180 located proximal to the first defibrillation coil 175, tip electrode 135, and ring electrode 140 for placement in the superior vena cava (SVC). In some examples, high-energy shock therapy is delivered from the first or RV coil 175 to the second or SVC coil 180. In some examples, the SVC coil 180 is electrically tied to an electrode formed on the hermetically-sealed IMD housing or can 150. This improves defibrillation by delivering current from the RV coil 175 more uniformly over the ventricular myocardium. In some examples, the therapy is delivered from the RV coil 175 only to the electrode formed on the IMD can 150. In some examples, the coil electrodes 175, 180 are used in combination with other electrodes for sensing signals.

Note that although a specific arrangement of leads and electrodes are shown the illustration, an IMD can be configured with a variety of electrode arrangements, including transvenous, endocardial, and epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes). The present methods and systems will work in a variety of configurations and with a variety of electrodes. Other forms of electrodes include meshes and patches which can be applied to portions of heart 105 or which can be implanted in other areas of the body to help “steer” electrical currents produced by IMD 110.

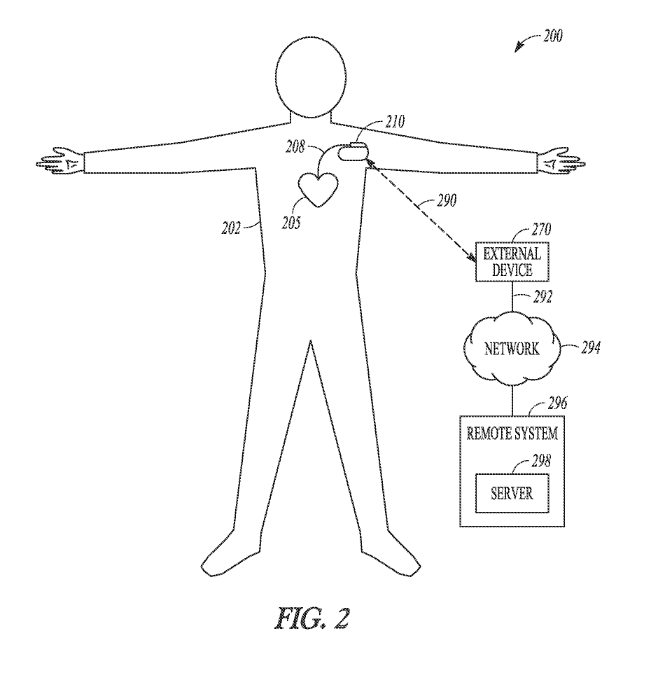

FIG. 2 is an illustration of portions of another system 200 that uses an IMD 210 to provide a therapy to a patient 202. The system 200 typically includes an external device 270 that communicates with a remote system 296 via a network 294. The network 294 can be a communication network such as a phone network or a computer network (e.g., the internet). In some examples, the external device includes a repeater and communicated via the network using a link 292 that may be wired or wireless. In some examples, the remote system 296 provides patient management functions and may include one or more servers 298 to perform the functions.

Providing pacing energy at an improper pacing site or location can lead to stow activation of myocardial tissue. Thus, it is desirable to have an IMD or other medical device that can automatically run tests to determine the best pacing site or sites, and either propose to the caregiver that these sites be used to provide the pacing therapy or automatically initiate delivery of pacing therapy to a determined optimal pacing site.

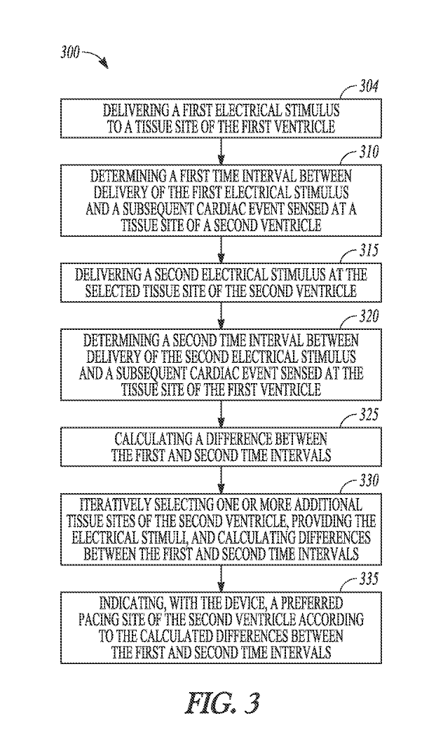

FIG. 3 is a flow diagram of an example of a method 300 of operating a medical device. At block 305, a first electrical stimulus is delivered to a tissue site of a first ventricle of a heart of a subject using the medical device. In some examples, the electrical stimulus includes sufficient energy to initiate a cardiac depolarization (e.g., a pacing therapy pulse).

At block 310, the medical device determines a first time interval between delivery of the first electrical stimulus and a subsequent cardiac event sensed at a selected tissue site of the second ventricle of the heart. The cardiac event can be any indication of cardiac activity that is of interest. For instance, a sensed cardiac event can include at least a portion of a cardiac depolarization, such as a sensed R-wave indicative of ventricular depolarization. Other cardiac events can include at least a portion of a QRS complex and a P-wave indicative of atrial depolarization.

At block 315, a second electrical stimulus is delivered, but at the selected tissue site of the second ventricle. At block 320, a second time interval is determined between delivery of the second electrical stimulus and a subsequent cardiac event sensed at the tissue site of the first ventricle. The subsequent or second sensed cardiac event may the same type of cardiac event (e.g., an R-wave) or can be a different cardiac event of interest.

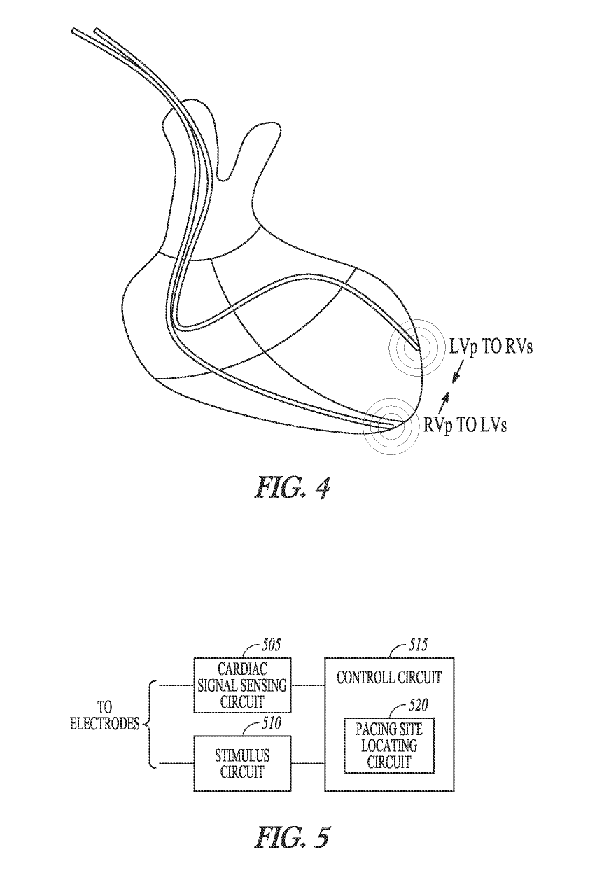

FIG. 4 illustrates an example of time intervals between cardiac events. In the example shown, the tissue site of the first ventricle is the apex of the right ventricle (RV). The tissue site of the second ventricle is an epicardial location of the left ventricle (IN). The first time interval is the time interval from an electrical stimulus at the tissue site of the RV (RVp) to a cardiac event sensed at the LV (LVs) or RVp−LVs. The second time interval is the time interval from an electrical stimulus at the tissue site of the LV (LVp) to a cardiac event sensed at the RV (RVs), or LVp−RVs.

At block 325, the medical device calculates a difference (Δ) between the first and second time intervals. For instance, in the example of FIG. 4, the device calculates the difference as Δ=(LVp−RVs)−(RVp−LVs).

At block 330, one or more additional tissue sites of the second ventricle are iteratively selected by the medical device and electrical stimuli is provided to the one or more sites. In some examples, the medical device iteratively selects a plurality of additional tissue sites and provides the stimuli. Differences between the first and second time intervals are calculated for the one or more sites. The first time intervals are the time intervals between delivery of an electrical stimulus provided at the site of the first ventricle and a subsequent cardiac event sensed at an one of the additional selected tissue sites of the second ventricle, and the second time intervals are between delivery of an electrical stimulus at one of the additional selected tissue sites of the second ventricle and a subsequent cardiac event sensed at the site of the first ventricle.

At block 335, a preferred pacing site of the second ventricle is indicated by the device according to the calculated differences between the first and second time intervals and the preferred pacing. In some examples, the preferred pacing site is the site of the second ventricle that results in the smallest difference in time intervals. The measured time intervals provide a bidirectional measure of propagation of the depolarization resulting from the stimulus (e.g., from RV to LV and LV to RV). A bidirectional measure may be more likely to uncover slow propagation paths or scar tissue than a unidirectional measure. The indication of the preferred pacing site can be provided to one or more of a user and a process.

FIG. 5 is a block diagram of portions of an example of a medical device 500 that can determine a preferred site to deliver pacing therapy. The device 500 includes a cardiac signal sensing circuit 505 configured to sense events related to cardiac activity of a subject and a stimulus circuit 510. The cardiac signal sensing circuit 505 can be electrically coupled to one or more cardiac electrodes to sense cardiac events, such as event related to cardiac depolarization for example.

The stimulus circuit 510 provides an electrical stimulus to a plurality of cardiac electrodes disposed in or near a heart of the subject. In some examples, the stimulus circuit 510 delivers electrical pacing therapy pulses.

The device 500 also includes a control circuit 515 communicatively coupled to the cardiac signal sensing circuit 505 and the stimulus circuit 510. The communicative coupling allows electrical signals to be communicated between the control circuit and one or both of the cardiac signal sensing circuit 505 and the stimulus circuit 510 even though there may be one or more intervening circuits between the control circuit 515 and the cardiac signal sensing circuit 505 and the stimulus circuit 510. For example, the device 500 may include a sampling circuit (not shown) integral to the control circuit 515 or electrically coupled between the cardiac signal sensing circuit 505 and the control circuit 515. The sampling circuit can sample a sensed cardiac signal to produce cardiac signal data.

The control circuit 515 can include a processor such as a microprocessor, a digital signal processor, application specific integrated circuit (ASIC), or other type of processor, interpreting or executing instructions in software modules or firmware modules. In some examples, the control circuit is a sequencer. A sequencer refers to a state machine or other circuit that sequentially steps through a fixed series of steps to perform one or more functions. The steps are typically implemented in hardware or firmware. The control circuit 515 includes other circuits or sub-circuits to perform the functions described. These circuits may include software, hardware, firmware or any combination thereof. Multiple functions can be performed in one or more of the circuits or sub-circuits as desired.

The control circuit 515 includes a pacing site locating circuit 520. The pacing site locating circuit 520 initiates a first electrical stimulus from a first electrode positioned in or near a first ventricle of a heart and measures or otherwise determines a first time interval between delivery of the first electrical stimulus and a subsequent cardiac event sensed at a selectable electrode location in or near a second ventricle of the heart. The pacing site locating circuit 520 then initiates a second electrical stimulus at the selectable electrode location in or near the second ventricle and determines a second time interval between delivery of the second electrical stimulus and a subsequent cardiac event sensed at the first ventricle.

The stimulus circuit can be electrically coupled to a variety of electrodes such as the electrodes shown in the arrangement of the example in FIG. 1. In some examples, the pacing site locating circuit 520 initiates the first electrical stimulus in the RV and initiates the second electrical stimulus in the LV. The selected tissue site of a second ventricle can include at least one of an endocardial, transvenous, and epicardial site of the LV. The pacing site locating circuit 520 finds an optimal pacing site in the LV of the subject. Conversely, in some examples, the pacing site locating circuit 520 initiates the first electrical stimulus in the LV and initiates the second electrical stimulus in the RV. The pacing site locating circuit 520 finds an optimal pacing site in the RV of the subject.

Bipolar pacing refers to delivering pacing energy using electrodes that are in proximity to each other (e.g., located in or near the same heart chamber, such as pacing between tip electrode 135 and ring electrode 140 or between ring electrodes 160 and 165 in FIG. 1). Unipolar pacing refers to delivering pacing energy using electrodes that are more remote from each other (e.g., a first electrode located in or near a heart chamber and a second electrode located on the housing of the IMD in FIG. 1).

In some examples, the pacing site locating circuit 520 initiates bipolar pacing in the first ventricle (e.g., RV) and initiates unipolar pacing in the second ventricle (e.g., LV). In certain examples, the device 500 is an implantable medical device, or IMD, and includes the cardiac signal sensing circuit 505, the stimulus circuit 510, and the control circuit 515, and a hermetically sealed housing. The pacing site locating circuit 520 initiates the first electrical stimulus between a bipolar electrode pair that includes the first electrode, and initiates the second electrical stimulus between the selectable electrode and a fourth electrode formed on the housing.

In some examples, the pacing site locating circuit 520 initiates unipolar pacing in both the first ventricle and the second ventricle. In certain examples, the pacing site locating circuit 520 initiates the first electrical stimulus between the first electrode on or near the first ventricle and an electrode formed on the housing, and initiates the second electrical stimulus between the selectable electrode on or near the second ventricle and the electrode formed on the housing.

Sensing by the device 500 can also be bipolar or unipolar. In some examples, both the sensing in the first ventricle and the sensing in the second ventricle includes bipolar sensing. In certain examples, the cardiac signal sensing circuit 505 senses the cardiac event subsequent to the first electrical stimulus using the selectable electrode as part of a bipolar electrode pair included in a lead designed for transvenous placement on the LV, and senses the cardiac event subsequent to the second electrical stimulus using a bipolar electrode pair that includes the first electrode in the first ventricle (e.g., the bipolar pair may be tip electrode 135 and ring electrode 140, or ring electrode 140 and coil electrode 175).

In some examples, sensing events in the first ventricle includes unipolar sensing and sensing events in the second ventricle includes bipolar sensing. In certain examples, the cardiac signal sensing circuit 505 senses the cardiac event subsequent to the first electrical stimulus using the selectable electrode in the second ventricle as part of a bipolar electrode pair included in a lead designed for transvenous placement on the LV, and senses the cardiac event subsequent to the second electrical stimulus using an electrode pair that includes the first electrode in or near the RV and an electrode formed on the housing.

In some examples, the first and second time intervals are determined from several cycles of pacing at the first tissue site and pacing at the second tissue site. In certain examples, the pacing site locating circuit 520 provides multiple (e.g., 15) stimuli from the site of the first ventricle and measures the time until the subsequent cardiac event at the selectable location in the second ventricle. The first time interval may then be determined using a. central tendency (e.g., mean or median) of the measurements. In certain examples, measurements that are outliers are discarded. The pacing site locating circuit 520 then provides the same or a different number of stimuli from the selectable location in the second ventricle and measures the time until the subsequent cardiac event at the first ventricle site. The second time interval may also be determined using a central tendency of the measurements, and again, outlier measurements can be discarded.

When the pacing site locating circuit 520 determines the values of the first and second intervals, it then calculates the difference between the first and second time intervals. The pacing site locating circuit 520 iteratively selects one or more additional electrodes located in or near the second ventricle, provides the electrical stimuli, determines the first and second time intervals, and calculates differences between determined sets of the first and second time intervals.

In an illustrative and non-limiting example, the pacing site locating circuit 520 may initiate a first electrical stimulus in the RV using the tip electrode 135 and ring electrode 140 of FIG. 1, and sense a cardiac event in the LV using electrodes 165 and 160. The pacing site locating circuit 520 may then initiate a second electrical stimulus using electrode 165 and an electrode formed on the housing 150. The pacing site locating circuit 520 then iteratively selects different electrode combinations using electrodes 160, 162, 164, and 165, provides the electrical stimuli, and determines the first and second intervals for each of the combinations. The pacing site locating circuit 520 then calculates differences between each determined set of first and second time intervals.

When the differences in the time intervals are calculated, the pacing site locating circuit 520 generates an indication of a preferred pacing site in the second ventricle according to the calculated differences between the first and second time intervals. In some examples, the pacing site locating circuit 520 generates the indication of a preferred pacing site that corresponds to a minimum difference between the first and second time intervals. This indicates the pacing site that results in the smallest difference in directional propagation of myopotentials in myocardial tissue. Ideally the difference between the first interval and the second interval for the preferred pacing site would be zero. However, depending on the activation site used in the LV, it is likely that the first and second interval times will be different due to slower activation of cells of the LV wall.

In some examples, the pacing site locating circuit 520 generates the indication of a preferred pacing site that corresponds to a difference between the first and second time intervals that satisfies a specified (e.g., programmed) difference threshold value. If it is desired to minimize the difference in activation between the RV and LV, the preferred pacing site can be determined as the pacing site where the difference between the first and second time intervals is less than the specified difference threshold value. A physician or caregiver may desire to have some difference in activation between the RV and LV. In this case, the preferred pacing site can be determined as the pacing site where the difference between the first interval and the second interval is greater than a specified difference threshold value. In some examples, the pacing site locating circuit 520 is configured to generate the indication of a preferred pacing site that corresponds to a difference between the first and second time intervals that is within a specified range of time interval difference values.

In some examples, the device 500 is a pacing system analyzer (PSA) external to the subject, and the pacing system analyzer includes the cardiac signal sensing circuit 505, the stimulus circuit 510, and the control circuit 515. The cardiac signal sensing circuit 505 and the stimulus circuit 510 are connectable to cardiac leads or implantable pacing guide wires. The leads or guide wires include electrodes for delivering electrical pacing therapy pulses to the subject. The pacing system analyzer can provide one or more of unipolar pacing, bipolar pacing, unipolar sensing, and bipolar sensing. To determine a pacing site, the PSA may be connectable to a cardiac lead or guide wire that can be placed in a first coronary vein 122 as shown in FIG. 1. When pacing sites are analyzed using the first coronary vein 122, the lead or guide wire can be moved to a second coronary vein 124 and additional sites can be analyzed. Thus, many tissue sites may be evaluated in this manner. In some examples, the selected tissue site of a second ventricle includes at least one of an endocardial, transvenous, and epicardial site of the LV, and the pacing site locating circuit 520 indicates at least one of the endocardial, transvenous, and epicardial site as the preferred pacing site of the LV.

In some examples, the cardiac signal sensing circuit 505 and the stimulus circuit 510 are included in an implantable medical device, and the control circuit 515 and the pacing site locating circuit 520 are included in an external device used to program the implantable medical device.

If the control circuit 515 and the pacing site locating circuit 520 are included in a programmer or PSA, the methods described herein to determine a preferred pacing site can be implemented at time of implant of an IMD that provides pacing therapy. If the control circuit 515 and the pacing site locating circuit 520 are included in an IMD, the methods can be implemented recurrently (e.g., according to a schedule) for automatic recurrent selection of the preferred pacing site throughout the period of use of the IMD.

The indication of a preferred pacing site can be provided to a user or process. If the device 500 includes a programmer or PSA, the preferred pacing site may be presented to a user on a display of the device. The user may then configure an IMD and/or arrange cardiac leads for pacing at the indicated preferred pacing site. If the device 500 is an IMD, the indication of the preferred pacing site may be communicated (e.g., via wireless telemetry) to a second device where the indication can be displayed.

In some examples, the device 500 is an IMD and the stimulus circuit 510 provides electrical pacing to the subject to treat bradycardia, or a slow heart rhythm. The indication of the preferred pacing site can be provided to the control circuit 515 which automatically selects the preferred site (e.g., enables one or more pacing vectors) for delivery of pacing energy.

Finding an optimal pacing site should be part of optimizing pacing therapy. Automaticity in finding the optimal site simplifies the site selection process, which may result in more caregivers locating the optimal site for their patients. Using bidirectional timing information may improve the likelihood of finding the optimal pacing site.

### Additional Notes

Example 1 includes subject matter (such as an apparatus) comprising a cardiac signal sensing circuit configured to sense events related to cardiac activity of a subject, a stimulus circuit configured to provide an electrical stimulus to a plurality of cardiac electrodes disposed in or near a heart of the subject, and a control circuit communicatively coupled to the stimulus circuit and the cardiac signal sensing circuit. The control circuit includes a pacing site locating circuit configured to initiate a first electrical stimulus from a first electrode positioned in or near a first ventricle of a heart, determine a first time interval between delivery of the first electrical stimulus and a subsequent cardiac event sensed at a selectable electrode location in or near a second ventricle of the heart, initiate a second electrical stimulus at the selectable electrode location in or near the second ventricle, determine a second time interval between delivery of the second electrical stimulus and a subsequent cardiac event sensed at the first ventricle, calculate a difference between the first and second time intervals, select one or more additional electrodes located in or near the second ventricle, provide the electrical stimuli, and calculate differences between the first and second time intervals, and generate an indication of a preferred pacing site in the second ventricle according to the calculated differences between the first and second time intervals.

In Example 2, the pacing site locating circuit of Example 1 can optionally be configured to generate the indication of a preferred pacing site that corresponds to a minimum difference between the first and second time intervals.

In Example 3, the pacing site locating circuit of one or any combination of Example 1 and 2 can optionally by configured to generate the indication of a preferred pacing site that corresponds to a difference between the first and second time intervals that is within a specified range of time interval difference values.

In Example 4, the pacing site locating circuit of one any combination of Example 1-3 can optionally be configured to generate the indication of a preferred pacing site that corresponds to a difference between the first and second time intervals that satisfies a specified difference threshold value.

In Example 5, the pacing site locating circuit of one or any combination of Examples 1-4 can optionally be configured to initiate the first electrical stimulus in the right ventricle (RV) and initiate the second electrical stimulus in the left ventricle (LV).

In Example 6, the subject matter of one or any combination of Examples 1-5 can optionally include a hermetically sealed housing. The pacing site locating circuit can optionally be configured to initiate the first electrical stimulus between a bipolar electrode pair that includes the first electrode, and initiate the second electrical stimulus between the selectable electrode and an electrode formed on the housing.

In Example 7, the subject matter of one or any combination of Examples 1-5 can optionally include a hermetically sealed housing. The pacing site locating circuit can optionally be configured to initiate the first electrical stimulus between the first electrode and an electrode formed on the housing, and initiate the second electrical stimulus between the selectable electrode and the electrode formed on the housing.

In Example 8, the cardiac signal sensing circuit of one or any combination of Examples 1-7 can optionally be configured to sense the cardiac event subsequent to the first electrical stimulus using the selectable electrode as part of a bipolar electrode pair included in a lead for transvenous placement on the LV, and sense the cardiac event subsequent to the second electrical stimulus using a bipolar electrode pair that includes the first electrode.

In Example 9, the subject matter of one or any combination of Examples 1-5 can optionally include a hermetically sealed housing. The cardiac signal sensing circuit can optionally be configured to sense the cardiac event subsequent to the first electrical stimulus using the selectable electrode as part of a bipolar electrode pair included in a lead for transvenous placement on the LV, and sense the cardiac event subsequent to the second electrical stimulus using an electrode pair that includes the first electrode and an electrode formed on the housing.

In Example 10, the cardiac signal sensing circuit, the stimulus circuit, and the control circuit of one or any combination of Examples 1-9 can optionally be included in an implantable medical device.

In Example 11, the cardiac signal sensing circuit, the stimulus circuit, and the control circuit of one or any combination of Examples 1-9 can optionally be included in a pacing system analyzer.

In Example 12, the cardiac signal sensing circuit and the stimulus circuit of one or any combination of Examples 1-9 can optionally be included in an implantable medical device, and the control circuit and the pacing site locating circuit of the Examples can be included in an external device used to program the implantable medical device.

Example 13 can include subject matter, or can optionally be combined with the subject matter of one or any combination of Examples 1-12 to include subject matter (such as a method, a means for performing acts, or a machine-readable medium including instructions that, when performed by the machine, cause the machine to perform acts) comprising delivering a first electrical stimulus to a tissue site of a first ventricle of a heart, determining a first time interval between delivery of the first electrical stimulus and a subsequent cardiac event sensed at a selected tissue site of a second ventricle of the heart, delivering a second electrical stimulus at the selected tissue site of the second ventricle, determining a second time interval between delivery of the second electrical stimulus and a subsequent cardiac event sensed at the tissue site of the first ventricle, calculating a difference between the first and second time intervals, selecting one or more additional tissue sites of the second ventricle, providing the electrical stimuli, and calculating differences between the first and second time intervals, and indicating, with the device, a preferred pacing site of the second ventricle according to the calculated differences between the first and second time intervals.

Such subject matter can include a means for delivering a first electrical stimulus to a tissue site of a first ventricle of a heart, illustrative examples of which can include a stimulus circuit configured to provide an electrical stimulus to a plurality of cardiac electrodes disposed in or near a heart of the subject and a pacing therapy circuit. Such subject matter can include a means for determining a first time interval between delivery of the first electrical stimulus and a subsequent cardiac event sensed at a selected tissue site of a second ventricle of the heart, illustrative examples of which can include a pacing site locating circuit. Such subject matter can include a means for delivering a second electrical stimulus at the selected tissue site of the second ventricle, illustrative examples of which can include a stimulus circuit and a pacing therapy circuit. Such subject matter can include a means for determining a second time interval between delivery of the second electrical stimulus and a subsequent cardiac event sensed at the tissue site of the first ventricle, illustrative examples of which can include a pacing site locating circuit. Such subject matter can include a means for calculating a difference between the first and second time intervals, illustrative examples of which can include a pacing site locating circuit. Such subject matter can include a means for iteratively selecting one or more additional tissue sites of the second ventricle, illustrative examples of which can include a pacing site locating circuit. Such subject matter can also include a means for indicating, with the device, a preferred pacing site of the second ventricle according to the calculated differences between the first and second time intervals, illustrative examples of which are a display on an external device and wireless telemetry.

In Example 14, the indicating a preferred pacing site of Example 13 can optionally include indicating a preferred pacing location corresponding to a minimum difference between the first and second time intervals.

In Example 15, the indicating a preferred pacing site of one or any combination of Examples 13-15 can optionally include indicating a preferred pacing location corresponding to a difference between the first and second time intervals that is within a specified range of time interval difference values.

In Example 16, the indicating a preferred pacing site of one or any combination of Examples 13-15 can optionally include indicating a preferred pacing location corresponding to a. difference between the first and second time intervals that satisfies a specified difference threshold value.

In Example 17, the determining the first time interval of one or any combination of Examples 13-16 can optionally include determining a first time interval between delivery of the first electrical stimulus at a tissue site of a right ventricle (RV) and a subsequent cardiac event sensed at a selected tissue site of a left ventricle (LV), and the determining the second time interval can optionally include determining a second time interval between delivery of the second electrical stimulus to the selected tissue site of the LV and a subsequent cardiac event sensed at the tissue site of the RV.

In Example 18, the delivering the first electrical stimulus of one or any combination of Examples 13-17 can optionally include delivering a first electrical stimulus using first and second electrodes shaped and sized for placement in a RV, wherein the first and second electrodes form a bipolar electrode pair. The delivering the second electrical stimulus in the Examples can optionally include delivering a second electrical stimulus using a third electrode included in a lead for transvenous placement on the LV and a fourth electrode formed on a housing of the device.

In Example 19, the subject matter of one or any combination of Examples 13-18 can optionally include sensing the cardiac event subsequent to the first electrical stimulus in a LV using first and second electrodes included in a lead for transvenous placement on the LV, and sensing the cardiac event subsequent to the second electrical stimulus using third and fourth electrodes shaped and sized for placement in a RV, wherein the third and fourth electrodes form a bipolar electrode pair.

In Example 20, the selected tissue site of the second ventricle of one or any combination of Examples 13-19 can optionally include at least one of an endocardial, transvenous, and epicardial site of the LV, and the indicating a preferred pacing site includes indicating the at least one of the endocardial, transvenous, and epicardial site as the preferred pacing site of the LV.

Example 21 can include, or can optionally be combined with any portion or combination of any portions of any one or more of Examples 1-20 to include, subject matter that can include means for performing any one or more of the functions of Examples 1-20, or a machine-readable medium including instructions that, when performed by a. machine, cause the machine to perform any one or more of the functions of Examples 1-20.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples,” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
a cardiac signal sensing circuit configured to sense events indicative of cardiac activity of a subject;
a stimulus circuit configured to provide an electrical stimulus to a plurality of cardiac electrodes disposed in or near a heart of the subject; and
a control circuit communicatively coupled to the stimulus circuit and the cardiac signal sensing circuit, wherein the control circuit includes a pacing site locating circuit configured to:
initiate a first electrical stimulus from a first electrode positioned in or near a first ventricle of a heart;
determine a first time interval between delivery of the first electrical stimulus and a subsequent cardiac event sensed at a selectable electrode location in or near a second ventricle of the heart;
initiate a second electrical stimulus at the selectable electrode location in or near the second ventricle;
determine a second time interval between delivery of the second electrical stimulus and a subsequent cardiac event sensed at the first ventricle;
calculate a difference between the first and second time intervals;
select one or more additional electrodes located in or near the second ventricle, provide the electrical stimuli, and calculate differences between the first and second time intervals; and
generate an indication of a preferred pacing site of the second ventricle according to the calculated differences between the first and second time intervals.

2. The apparatus of claim 1, wherein the pacing site locating circuit is configured to generate the indication of a preferred pacing site that corresponds to a minimum difference between the first and second time intervals.

3. The apparatus of claim 1, wherein the pacing site locating circuit is configured to generate the indication of a preferred pacing site that corresponds to a difference between the first and second time intervals that is within a specified range of time interval difference values.

4. The apparatus of claim 1, wherein the pacing site locating circuit is configured to generate the indication of a preferred pacing site that corresponds to a difference between the first and second time intervals that satisfies a specified difference threshold value.

5. The apparatus of claim 1, wherein the pacing site locating circuit is configured to initiate the first electrical stimulus in the right ventricle (RV) and initiate the second electrical stimulus in the left ventricle (LV).

6. The apparatus of claim 5, including:
a header to receive a cardiac lead;
a hermetically sealed housing,
wherein the pacing site locating circuit is configured to:
initiate the first electrical stimulus between a bipolar electrode pair that includes the first electrode; and
initiate the second electrical stimulus between the selectable electrode and an electrode formed on at least one of the header or the housing.

7. The apparatus of claim 5, including:
a header to receive a cardiac lead;
a hermetically sealed housing,
wherein the pacing site locating circuit is configured to:
initiate the first electrical stimulus between the first electrode and an electrode formed on at least one of the header or the housing; and
initiate the second electrical stimulus between the selectable electrode and the electrode formed on the at least one of the header or the housing.

8. The apparatus of claim 5, wherein the cardiac signal sensing circuit is configured to:
sense the cardiac event subsequent to the first electrical stimulus using the selectable electrode as part of a bipolar electrode pair included in a lead for transvenous placement on the LV; and
sense the cardiac event subsequent to the second electrical stimulus using a bipolar electrode pair that includes the first electrode.

9. The apparatus of claim 5, including:
a header to receive a cardiac lead;
a hermetically sealed housing,
wherein the cardiac signal sensing circuit is configured to:
sense the cardiac event subsequent to the first electrical stimulus using the selectable electrode as part of a bipolar electrode pair included in a lead for transvenous placement on the LV; and
sense the cardiac event subsequent to the second electrical stimulus using an electrode pair that includes the first electrode and an electrode formed on at least one of the header or the housing.

10. The apparatus of claim 1, wherein the cardiac signal sensing circuit, the stimulus circuit, and the control circuit are included in an implantable medical device.

11. The apparatus of claim 1, wherein the cardiac signal sensing circuit, the stimulus circuit, and the control circuit are included in a pacing system analyzer.

12. The apparatus of claim 1, wherein the cardiac signal sensing circuit and the stimulus circuit are included in an implantable medical device and control circuit and the pacing site locating circuit are included in an external device used to program the implantable medical device.

13. A method of operating a medical device, the method comprising:
delivering a first electrical stimulus to a tissue site of a first ventricle of a heart;
determining a first time interval between delivery of the first electrical stimulus and a subsequent cardiac event sensed at a selected tissue site of a second ventricle of the heart;
delivering a second electrical stimulus at the selected tissue site of the second ventricle;
determining a second time interval between delivery of the second electrical stimulus and a subsequent cardiac event sensed at the tissue site of the first ventricle;
calculating a difference between the first and second time intervals;
selecting one or more additional tissue sites of the second ventricle, providing the electrical stimuli, and calculating differences between the first and. second time intervals; and
indicating, with the device, a preferred pacing site of the second ventricle according to the calculated differences between the first and second time intervals.

14. The method of claim 13, wherein indicating a preferred pacing site includes indicating a preferred pacing location corresponding to a minimum difference between the first and second time intervals.

15. The method of claim 13, wherein indicating a preferred pacing site includes indicating a preferred pacing location corresponding to a difference between the first and second time intervals that is within a specified range of time interval difference values.

16. The method of claim 13, wherein indicating a preferred pacing site includes indicating a preferred pacing location corresponding to a difference between the first and second time intervals that satisfies a specified difference threshold value.

17. The method of claim 13,
wherein determining the first time interval includes determining a first time interval between delivery of the first electrical stimulus at a tissue site of a right ventricle (RV) and a subsequent cardiac event sensed at a selected tissue site of a left ventricle (LV), and
wherein determining the second time interval includes determining a second time interval between delivery of the second electrical stimulus to the selected tissue site of the LV and a subsequent cardiac event sensed at the tissue site of the RV.

18. The method of claim 13,
wherein delivering the first electrical stimulus includes delivering a first electrical stimulus using first and second electrodes shaped and sized for placement in a RV, wherein the first and second electrodes form a bipolar electrode pair, and
wherein delivering the second electrical stimulus includes delivering a second electrical stimulus using a third electrode included in a lead for transvenous placement on the LV and a fourth electrode formed on a housing of the device.

19. The method of claim 13, including
sensing the cardiac event subsequent to the first electrical stimulus in a LV using first and second electrodes included in a lead for transvenous placement on the LV; and
sensing the cardiac event subsequent to the second electrical stimulus using third and fourth electrodes shaped and sized for placement in a RV, wherein the third and fourth electrodes form a bipolar electrode pair.

20. The method of claim 13,
wherein the selected tissue site of the second ventricle includes at least one of an endocardial, transvenous, and epicardial site of the LV, and
wherein indicating a preferred pacing site includes indicating the at least one of the endocardial, transvenous, and epicardial site as the preferred pacing site of the LV.
